(19) United States
(12) Patent Application Publication
(10) Pub. No.: US 2017/0202532 A1
(43) Pub. Date: Jul. 20, 2017

(54) DATA PROCESSING METHOD, DATA PROCESSING DEVICE, AND X-RAY CT APPARATUS

(71) Applicant: HITACHI, LTD., Tokyo (JP)

(72) Inventors: Taiga GOTO, Tokyo (JP); Hisashi TAKAHASHI, Tokyo (JP); Koichi HIROKAWA, Tokyo (JP)

(73) Assignee: HITACHI, LTD., Tokyo (JP)

(21) Appl. No.: 15/321,401

(22) PCT Filed: Jul. 10, 2015
(86) PCT No.: PCT/JP2015/069863

§ 371 Date: Dec. 22, 2016

(30) Foreign Application Priority Data
Jul. 30, 2014 (JP) 2014-155062

(51) Int. Cl. A61B 6/00; A61B 6/04; G06T 11/00; A61B 6/03

(52) U.S. Cl. CPC A61B 6/5294; A61B 6/032; A61B 6/545; A61B 6/0457; G06T 11/003

(57) ABSTRACT

In order to provide a data processing method and the like capable of suppressing high-frequency errors such as moiré using data uniformly by presuming that adjacent pixels and beams overlap and performing calculation in a back projection process or a forward projection process for reconstructing images, the image processing device 122 sets a pixel size wider than a pixel interval, performs the back projection process or the forward projection process for calculating an interpolation value to be assigned to the pixels or the beams using a size-dependent weight (pixel window) according to an overlap amount of the adjacent pixels, sets a beam size wider than a beam interval, and then performs the back projection process or the forward projection process for calculating an interpolation value to be assigned to the pixels or the beams using a size-dependent weight (beam window) according to an overlap amount of the adjacent beams.

### TECHNICAL FIELD

The present invention relates to a data processing method, a data processing device, and an X-ray CT apparatus and, in detail, to forward projection and back projection processes in an image reconstruction process.

### BACKGROUND ART

Conventionally, an analytical method and a successive approximation method such as a filter correction back projection method are used as a method for reconstructing tomographic images from measurement data acquired by an X-ray CT (Computed Tomography) apparatus or the like. For example, in a successive approximation reconstruction method, a likely image is estimated in a successive approximation manner by repeating a back projection process that generates an image from projection data and a forward projection process that performs line integral on the projection line from an image by the predetermined number of repetitions. (1) ray-driven method, (2) pixel-driven method, and (3) distance-driven method are suggested as the back projection process and the forward projection process to be performed in these image reconstruction processes.

(1) Forward projection and back projection processes of the beam-driven method are methods that use beams as references and scan the beams to embed projection values in pixels that contribute to each beam sequentially.

(2) Forward projection and back projection processes of the pixel-driven method are methods that use pixels as references and scan the pixels to embed projection values related to each pixel sequentially.

(3) Forward projection and back projection processes of the distance-driven method are methods that use distances between pixel boundaries and beam boundaries as references and scan the distanced between pixel boundaries and beam boundaries to embed projection values in the pixels included in the beams sequentially.

The above beam-driven method treats beams as line segments and assigns (performs back projection on) values of projection data (projection values) to pixels through which the line segments pass. Therefore, there are some pixels to which the projection values were not assigned in a case of narrow pixel intervals, which results in uneven sampling. The uneven sampling is a problem, causing moire or the like that appears on images. In a case of adopting the pixel-driven method, pixels are focused in order to assign values of beams (projection data) passing through the pixel center of a target pixel. Therefore, unused projection data is left in a case of coarse pixels. Then, the usage efficiency of the projection data is reduced, which results in much image noise. Also, in the pixel-driven method and the beam-driven method, pixels (beams) are used or not used depending on an angle at which back projection is performed, which causes uneven processing. If this is repeated successively, high-frequency errors occur.

In contrast to this, it is possible to keep sampling density constant in a case of adopting back projection and forward projection of the distance-driven method. Patent Literature 1 describes projection and back projection methods that dynamically adjust a dimension of a square window for one of a pixel and a detector bin so that adjacent windows form a continuous shadow over one of the detector bin and the pixel and determine the effect of each pixel on each bin of the detector or vice versa. According to PTL 1, noise is reduced in a case where a pixel size is relatively larger compared to a detector element size, which enables uniform back projection. This has an advantage that high-frequency errors such as moiré do not occur.

### CITATION LIST

### Patent Literature

### PTL 1: Japanese Unexamined Patent Application Publication No. 2005-522304

### SUMMARY OF INVENTION

### Technical Problem

However, in the above PTL 1, adjacent windows are arranged continuously. That is, a pixel size is set equally to a pixel interval. In this case, image quality can be deteriorated due to aliasing in a case of performing 3D display such as volume rendering. Also, in a case where a structure with a size equivalent to or smaller than the pixel size is located between pixels, this causes a problem that drawing ability is reduced due to a partial volume effect. Also, in a case of adopting the back projection described in PTL 1 in the successive approximation reconstruction method, much noise is generated due to lack of X-ray photons after thinning a slice thickness, which may not be able to obtain desired image quality. On the other hand, when the slice thickness is thickened, a smoothing process (regularization process) based on similarity of adjacent pixels in an image space, which is performed in the successive approximation reconstruction process, does not work well, and this can cause deterioration in drawing minute structures. In order to avoid such a phenomenon, it is better to thicken the slice thickness according to a scanning dose, and a distance between pixels had better not be extremely far. Consequently, it is desirable to set the pixel interval narrower than the pixel size. In other words, it is desirable to set a pixel interval and a pixel size at which adjacent pixels overlap.

The present invention was made in light of the above problems and has a purpose to provide a data processing method, a data processing device, and an X-ray CT apparatus capable of suppressing occurrence of high-frequency errors such as moiré using data uniformly by presuming that adjacent pixels and beams overlap and performing calculation taking overlap of the pixels and the beams into account in a back projection process or a forward projection process to be performed in an image reconstruction process.

### Solution to Problem

In order to achieve the above purpose, the present invention is a data processing method characterized by setting a beam size to be set wider than a beam interval or a pixel size wider than a pixel interval in a forward projection process or a back projection process to be executed by a data processing device and calculating an interpolation value to be assigned to the beams or the pixels using a size-dependent weight according to an overlap amount of the adjacent beams or an overlap amount of the adjacent pixels.

Also, the present invention is an X-ray CT apparatus having a data processing device and a data processing device characterized by comprising a setting unit that sets a beam size to be set wider than a beam interval or a pixel size wider than a pixel interval in a forward projection process or a back projection process and a calculation unit that calculates an interpolation value to be assigned to the beams or the pixels using a size-dependent weight according to an overlap amount of the adjacent beams or an overlap amount of the adjacent pixels.

Additionally, the present invention is an X-ray CT apparatus characterized by comprising an X-ray source that irradiates X-rays from the focus with an area; an X-ray detector that is disposed opposite to the X-ray source and detects X-rays transmitted through an object; a data acquisition device that acquires transmission X-rays detected by the X-ray detector; and an image processing device that obtains the transmission X-rays and executes an image reconstruction process that includes a process for setting a beam size wider than a beam interval in a forward projection process or a back projection process to reconstruct an image based on the obtained transmission X-rays and calculating an interpolation value to be assigned to the beams or the pixels using a size-dependent weight according to an overlap amount of the adjacent beams.

### Advantageous Effects of Invention

The present invention can provided a data processing method, a data processing device, and an X-ray CT apparatus capable of suppressing occurrence of high-frequency errors such as moiré and using data uniformly to evaluate a value to be assigned to pixels or beams taking overlap of the pixels and the beams into account by presuming that adjacent pixels and beams overlap in a back projection process or a forward projection process for reconstructing images.

### BRIEF DESCRIPTION OF DRAWINGS

FIG. 1 illustrates an overall configuration of an X-ray CT apparatus 1.

FIG. 2 shows examples of pixel windows and beam windows to be used for the back projection process or the forward projection process related to the present invention (beam window width bww>pixel window width pww).

FIG. 3 shows examples of pixel windows and beam windows to be used for the back projection process or the forward projection process related to the present invention (beam window width bww<pixel window width pww).

FIG. 4 illustrates a flow chart of a procedure for calculating a value to be assigned to a pixel pc (a beam interpolation value pv) in the back projection process using the pixel windows illustrated in FIGS. 2 and 3.

FIG. 5 illustrates a flow chart of a procedure for calculating a value to be assigned to a beam bc (a pixel interpolation value by) in the forward projection process using the pixel windows illustrated in FIGS. 2 and 3.

FIG. 6 illustrates a general beam window.

FIG. 7 illustrates a relationship between beam intervals and beam widths in the present invention (beam intervals<beam widths) and a beam window according to a distance from the ray source.

FIG. 8 shows examples of pixel windows and beam windows to be used for the back projection process or the forward projection process related to the present invention (beam window width bww<pixel window width pww).

FIG. 9 shows examples of pixel windows and beam windows to be used for the back projection process or the forward projection process related to the present invention (beam window width bww>pixel window width pww).

FIG. 10 illustrates a flow chart of a procedure for calculating a value to be assigned to a pixel pc (a beam interpolation value pv) in the back projection process using the beam windows illustrated in FIGS. 8 and 9.

FIG. 11 illustrates a flow chart of a procedure for calculating a value to be assigned to a beam bc (a pixel interpolation value by) in the forward projection process using the beam windows illustrated in FIGS. 8 and 9.

FIG. 12 illustrates a flow chart of a procedure for calculating a beam interpolation value pv in back projection in which overlap between adjacent beams and overlap between adjacent pixels are taken into account.

FIG. 13 illustrates a flow chart of a procedure for calculating a pixel interpolation value by in forward projection in which overlap between adjacent beams and overlap between adjacent pixels are taken into account.

FIG. 14(a) illustrates a dose distribution (electronic density distribution) at the radiation source, and FIG. 14(b) illustrates a sensitivity distribution of the X-ray detector.

### DESCRIPTION OF EMBODIMENTS

Hereinafter, each embodiment of the present invention will be described in detail referring to the drawings.

### First Embodiment

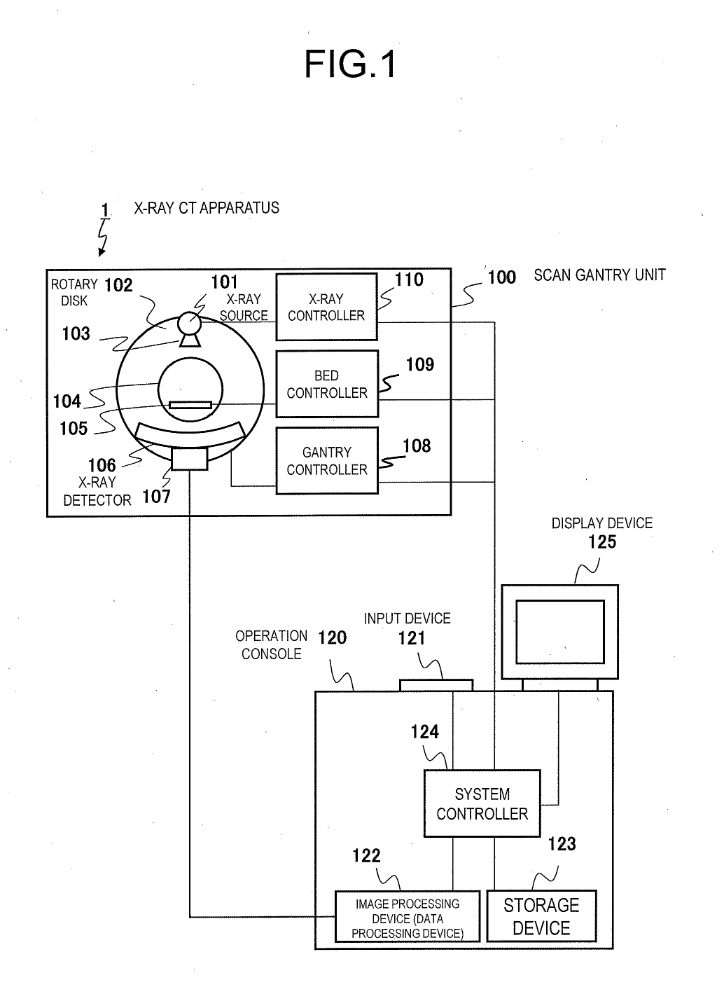

First, the overall configuration of the X-ray CT apparatus 1 will be described referring to FIG. 1.

As illustrated in FIG. 1, the X-ray CT apparatus 1 comprises a scan gantry unit 100, a bed 105, and an operation console 120. The scan gantry unit 100 is a device that irradiates X-rays to an object and detects the X-rays transmitted through the object. The operation console 120 is a device that controls each part of the scan gantry unit 100 and acquires the transmission X-ray data measured by the scan gantry unit 100 in order to generate images. The bed 105 is a device for placing the object and carrying the object in/from an X-ray irradiation range of the scan gantry unit 100.

The scan gantry unit 100 comprises an X-ray source 101, a rotary disk 102, a collimator 103, an X-ray detector 106, a data acquisition device 107, a gantry controller 108, a bed controller 109, and an X-ray controller 110.

The operation console, 120 comprises an input device 121, an image processing device (data processing device) 122, a storage device 123, a system controller 124, and display device 125.

The rotary disk 102 of the scan gantry unit 100 is provided with an opening 104, and the X-ray source 101 and the X-ray detector 106 are disposed opposite to each other across the opening 104. The object placed on the bed 105 is inserted in the opening 104. The rotary disk 102 rotates around the periphery of the object by a driving force transmitted through a driving transmission system from a rotary disk driving device. The rotary disk driving device is controlled by the gantry controller 108.

The X-ray source 101 is controlled by the X-ray controller 110 to continuously or intermittently irradiate X-rays at a predetermined intensity. The X-ray controller 110 controls an X-ray tube voltage and an X-ray tube current to be applied or supplied to the X-ray source 101 according to the X-ray tube voltage and the X-ray tube current determined by the system controller 124 of the operation console 120.

The X-ray irradiation port of the X-ray source 101 is provided with the collimator 103. The collimator 103 limits an irradiation range of X-rays emitted from the X-ray source 101. For example, the irradiation range is shaped into a cone beam (cone- or pyramid-shaped beam) or the like. The opening width of the collimator 103 is controlled by the system controller 124.

X-rays are irradiated from the X-ray source 101, pass through the collimator 103, transmit through an object, and enter the X-ray detector 106.

The X-ray detector 106 is a detector in which X-ray detection element groups composed of, for example, combination of a scintillator and a photodiode are two-dimensionally arranged in a channel direction (circumferential direction) and a column direction (body-axis direction). The X-ray detector 106 is disposed so as to be opposite to the X-ray source 101 across an object. The X-ray detector 106 detects amounts of X-rays irradiated from the X-ray source 101 and transmitted through the object and outputs the amount to the data acquisition device 107.

The data acquisition device 107 acquires the X-ray amounts to be detected by each X-ray detection element of the X-ray detector 106 at predetermined sampling intervals, converts the amounts into digital signals, and sequentially outputs them to the image processing device 122 of the operation console 120 as transmission X-ray data.

The image processing device (data processing device) 122 acquires transmission X-ray data input from the data acquisition device 107, performs preprocesses including logarithmic transformation and sensitivity correction, and then generates projection data necessary for reconstruction. Also, the image processing device 122 reconstructs object images such as tomographic images using the generated projection data. The system controller 124 stores the object image data reconstructed by the image processing device 122 in the storage device 123 and displays the data on the display device 125.

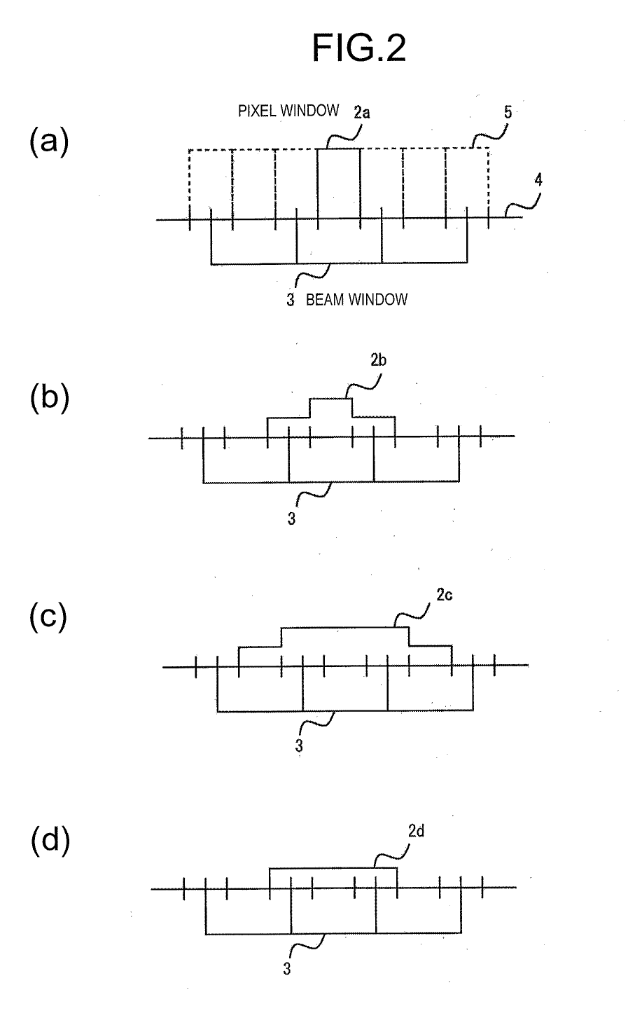

In an image reconstruction process to be executed by the image processing device 122 in the first embodiment, performed is a back projection process that includes a process for setting a pixel size wider than a pixel interval and calculating an interpolation value to be assigned to the pixels using a size-dependent weight (pixel window) according to an overlap amount between the adjacent pixels. The details of the back projection process will be described later (refer to FIGS. 2 to 4).

The system controller 124 is a computer comprising a CPU (Central Processing Unit), a ROM (Read Only Memory), a RAM (Random Access Memory), and the like. The storage device 123 is a data recording device such as a hard disk and previously stores a program, data, and the like to realize functions of the X-ray CT apparatus 1.

The display device 125 comprises a display device such as a liquid-crystal panel and a CRT monitor and a logical circuit for executing a display process in association with the display device and is connected to the system controller 124. The display device 125 displays object images to be output from the image processing device 122 and various information to be handled by the system controller 124.

The input device 121 is composed of, for example, a pointing device such as a keyboard and a mouse, a numeric key pad, various switch buttons, and the like and outputs various commands and information to be input by an operator to the system controller 124. The operator interactively operates the X-ray CT apparatus 1 using the display device 125 and the input device 121. The input device 121 may be a touch panel-type input device integrally configured with the display device 125.

The bed 105 comprises a top plate for placing an object, a vertical movement device, and a top plate driving device, vertically adjusts the height of the top plate under control of the bed controller 109, moves back and forth in the body-axis direction, and horizontally moves in a direction vertical to the body axis and a direction parallel to the floor (horizontal direction). During scanning, the bed controller 109 moves the top plate at a bed moving speed in a moving direction determined by the system controller 124.

Next, referring to FIGS. 2 to 4, described will be a back projection process for image reconstruction in the image processing device 122 related to the present invention. For example, in a back projection process for reconstructing images using an analytical method such as a filter correction three-dimensional back projection method, the image processing device 122 sets a pixel size wider than a pixel interval. This causes overlap between adjacent pixels. The image processing device 122 calculates a size-dependent weight (pixel window) according to an overlap amount between the adjacent pixels and calculates an interpolation weight to be assigned to pixels using the size-dependent weight (pixel window).

First, scanning conditions and reconstruction conditions are input from the input device 121 of the X-ray CT apparatus 1 before scanning an object. The scanning conditions are set so as to be, for example, a beam pitch: 1.1, a tube voltage: 120 kV, a tube current 300 mA, a scan speed: 0.5 s/rotation. Also, a reconstruction FOV (Field Of View) and a reconstruction center position included in the reconstruction conditions are determined so as to easily diagnose diseases according to the scanning site. For example, in scanning of the heart, the reconstruction FOV is set to “250 mm”, and the reconstruction center position is set as “the heart is at the center”.

Also, a reconstructed image matrix size is normally fixed at 512 pixels (the number of pixels on a side of a square reconstruction image), and the number of reconstructed image slices, the slice interval, and the slice thickness are set according to a scanning range, a size of a disease to be diagnosed, and a scanning dose. For example, the number of slices is set to 200 pieces, the slice interval is set to 1.25 mm, and the slice thickness is set to 2.5 mm. Also, a reconstruction filter is selected according to a scanning site. For example, “Standard Filter for Abdomen” may be selected in scanning of the abdomen, and “Standard Filter for Head” may be selected in scanning of the head.

The image processing device 122 acquires projection data by scanning and executes an image reconstruction process based on the above reconstruction conditions in order to generate reconstruction images. For example, the filter correction three-dimensional back projection method is used for the image reconstruction method. In the filter correction three-dimensional back projection method, the image processing device 122 performs a back projection process taking overlap between adjacent pixels into account. Hereinafter, referring to FIGS. 2 to 4, described will be the back projection process for which the overlap between adjacent pixels is taken into account.

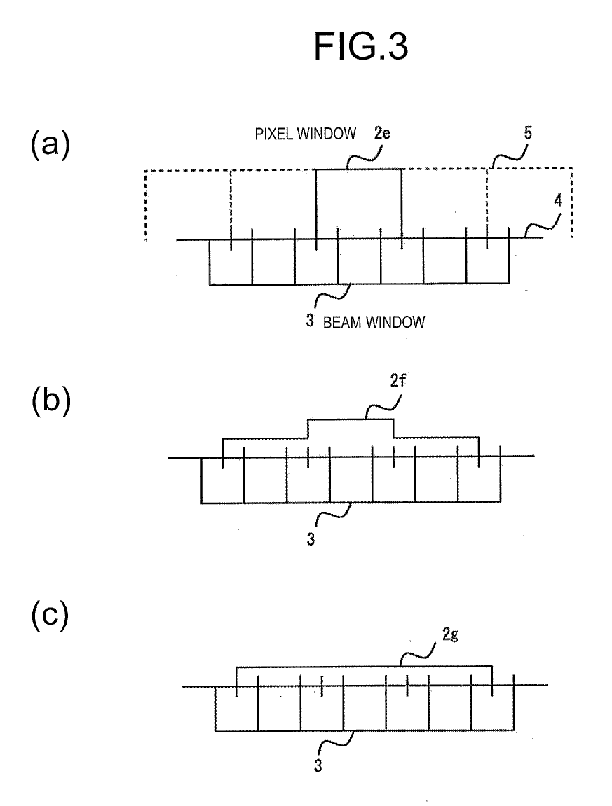

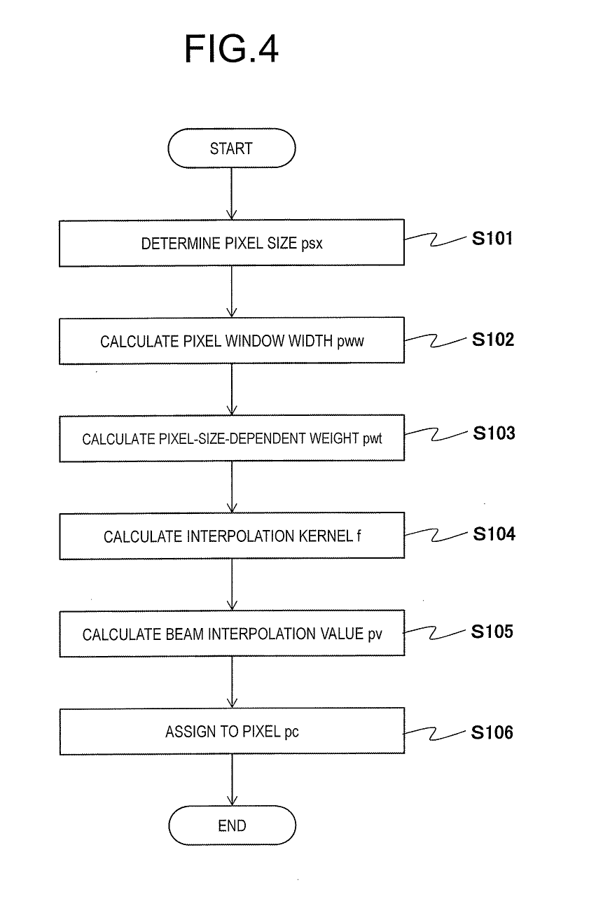

FIGS. 2 and 3 illustrate size-dependent weights (pixel windows 2a to 2g and a beam window 3) to be used for a back projection process in the present invention. FIG. 4 is a flow chart showing a processing procedure for calculating a value pv to be assigned to a pixel pc in the back projection process. It is noted that the pixel windows 2a to 2g are collectively referred to as a pixel window 2 in the following description.

The pixel window 2 is a weight to be used for calculating an interpolation value to be assigned to a pixel in the back projection process (size-dependent weight). The pixel window 2 to be used is determined according to an overlap amount of adjacent pixels. The shape of the pixel window 2 is defined by a width of the pixel window 2 (pixel window width pww) and a size of weights in each position (pixel region) in the width direction (pixel-size-dependent weight value pwtₖ). A length in the vertical direction of each of the pixel windows 2a to 2g illustrated in FIGS. 2 and 3 shows the pixel-size-dependent weight value pwtₖ. k is an index (a number that indicates the order of pixel regions from the left, such as 0, 1, 2, . . . ). The pixel regions are the respective regions in which pixels were segmented at a pixel interval.

In the present invention, the image processing device 122 determines a pixel window width pww from a pixel size (pixel size psx) and a pixel interval ppx. Additionally, a pixel-size-dependent weight values pwtₖ is determined so that the sum of weight values (pixel-size-dependent weight values pwtₖ) when adjacent pixel windows 2 are overlapped and arranged is equal in each pixel position and a half-value width of the pixel window 2 is equal to a pixel size.

FIG. 2 illustrates arrangements of the pixel windows 2 and the beam windows 3 in a case where a pixel size psx is wider than a pixel interval and a beam window width bww is greater than a pixel window width pww, (a) illustrates a shape of the pixel window 2a in a case where the pixel window width pww is equal to the pixel interval, (b) illustrates a shape of the pixel window 2b in a case where the pixel window width pww is double the pixel interval, (c) illustrates a shape of the pixel window 2c in a case where the pixel window width pww is four times as wide as the pixel interval, and (d) illustrates a shape of the pixel window 2d in a case where the pixel window width pww is three times as wide as the pixel interval.

Also, FIG. 3 illustrates arrangements of the pixel windows 2 and the beam windows 3 in a case where a pixel size psx is wider than a pixel interval and a beam window width bww is less than a pixel window width pww, (a) illustrates a shape of the pixel window 2e in a case where the pixel window width pww is equal to the pixel interval, (b) illustrates a shape of the pixel window 2f in a case where the pixel window width pww is double the pixel interval, and (c) illustrates a shape of the pixel window 2g in a case where the pixel window width pww is three times as wide as the pixel interval.

In the first embodiment, the pixel window 2 as illustrated in FIG. 2 or 3 is used for evaluating a beam interpolation value pv to be assigned to each pixel in the back projection process. Which pixel window 2 is used is determined according to an overlap amount between pixels. For example, the overlap amount between pixels is determined according to a relationship between a slice thickness and a slice interval.

Hereinafter, referring to the flow chart of FIG. 4, a procedure for calculating a beam interpolation value pv will be by described taking an overlap amount between adjacent pixels into account.

As illustrated in the flow chart of FIG. 4, the image processing device 122 evaluates the pixel window 2 when a pixel size psx [mm] is determined under reconstruction conditions and the like set by an operator through the input device 121 or the like (Step S101). That is, a pixel window width pww and a pixel-size-dependent weight value pwtₖ are calculated (Steps S102 and S103).

When an effective visual field size is FOV and a matrix size of a reconstruction image is MATRIX, a pixel interval ppx is calculated using the following equation (1).

$$ppx = \frac{FOV}{MATRIX} \qquad ( 1 )$$

It is noted that, for example, the above pixel size psx is used as a slice thickness of an reconstruction image and the pixel interval ppx is used as a slice interval of the reconstruction image.

When the pixel size determined in Step S101 is set as psx [mm], the image processing device 122 evaluates a pixel window width pww [pixel] using the following equation (2) (Step S102).

$$pww = ⌈ \frac{psx - ppx}{2 \cdot ppx} ⌉ \cdot 2 + 1 \qquad ( 2 )$$

It is noted that a pixel window central position pwc at a pixel window width pww is expressed by the equation (3) and that a pixel window end position pwe for the pixel window central position pwc is expressed by the equation (4).

$$\begin{matrix}pwc = \frac{pww - 1}{2} & ( 3 ) \\ pwe = \pm ⌈ \frac{psx}{2 \cdot ppx} ⌉ & ( 4 )\end{matrix}$$

Next, the image processing device 122 segments pixels at a pixel interval to determine a pixel-size-dependent weight value pwtₖ in each of the segmented pixel region. That is, the following equation (5) is used for calculating a pixel-size-dependent weight value pwtₖ of the k-th pixel from the left end pixel (pixel of k=0) in the pixel window 2 (Step S103).

$${pwt}_{k} = \{ \begin{matrix}\frac{\frac{psx - ppx}{2 \cdot ppx} - ⌊ \frac{psx - ppx}{2 \cdot ppx} ⌋}{psx} & if k = pwc + pwe \\ \frac{1}{psx} & if pwc - pwe < k < pwc + pwe\end{matrix} \qquad ( 5 )$$

A leading pixel position psc of the pixel window 2 is expressed by the following equation (6).

$$psc = - ⌈ \frac{psx - ppx}{2 \cdot ppx} ⌉ \qquad ( 6 )$$

Next, the image processing device 122 calculates an interpolation kernel f (Step S104) and calculates a beam interpolation value pv (Step S105). Hereinafter, the calculation of the interpolation kernel f and the beam interpolation value pv will be described.

In a case of using a pixel size as a pixel interval and a beam size as a beam interval, a beam interpolation value pvⱼ to be assigned to a pixel pcⱼ is calculated by the following equations (7) and (8) by setting as follows: positions of pixel boundaries psⱼ and peⱼ of a pixel pcⱼ (j is a pixel index) on a common axis 4: P(psⱼ) and P(peⱼ), positions of beam boundaries bsᵢ and beᵢ of a beam bcᵢ (i is a beam index) on the common axis 4: P(bsᵢ) and P(beᵢ), beams in which the pixel boundaries P(psᵢ) and P(peᵢ) on the common axis 4 are located: bcⱼₛ and bcⱼₑ, an interpolation kernel, i.e. a rate at which the beam bcᵢ occupies the pixel pcⱼ on the common axis 4 (rate of the length on the common axis 4): f_{i, j}, and a projection value located in a position i on the common axis 4: rawᵢ.

$$\begin{matrix}{yy}_{j} = {\sum }_{i = js}^{je} {f}_{i , j} \cdot {raw}_{i} & ( 7 ) \\ {pv}_{j} = {\sum }_{k = 0}^{pww - 1} {pwt}_{k} \cdot {yy}_{j + psc + k} & ( 8 )\end{matrix}$$

The image processing device 122 assigns the above beam interpolation value pvⱼ to the pixel pcⱼ (Step S106).

As described above, the image processing device 122 sets a pixel size wider than a pixel interval and performs a back projection process taking into account pixel overlap in a case of reconstructing images using an analytical method such as a filter correction three-dimensional back projection method in the first embodiment. Hence, satisfactory image quality with excellent data usage efficiency can be acquired without image quality deterioration caused by data usage inefficiency in a case of reconstructing images using the analytical method.

When a pixel size is used as a slice thickness of a reconstruction image and a pixel interval is used as a slice interval of the reconstruction image, a slice thickness wider than a slice interval is set, which can perform a back projection process. Because the slice thickness can be set wider than the slice interval of a reconstruction image, aliasing artifacts can be reduced during 3D display.

Also, a size-dependent weight is determined from the pixel size, pixels are segmented at a pixel interval, a size-dependent weight value is determined for the segmented pixel regions, and an interpolation value is calculated from the size-dependent weight and an interpolation kernel, which can reduce noise increase in a case where the pixel size is large. Also, data usage inefficiency can be reduced even in a case where the pixel size is set wider than the pixel interval.

### Second Embodiment

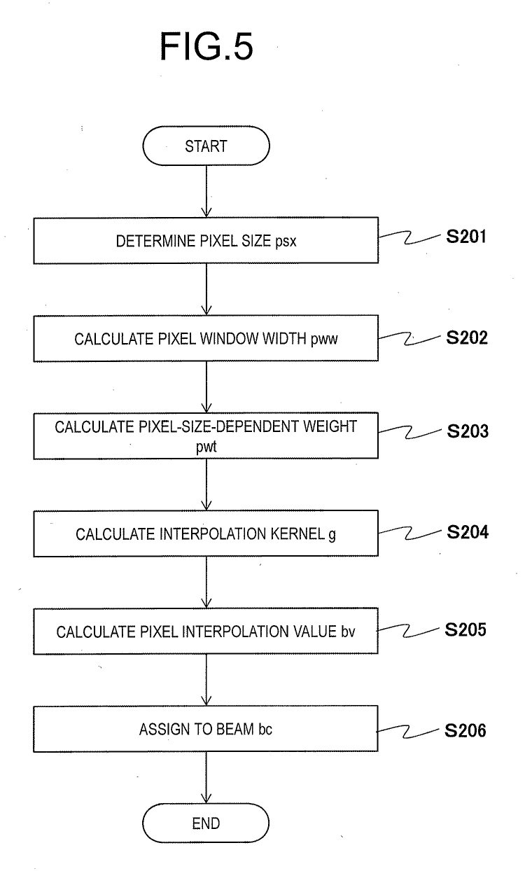

Next, a second embodiment of the present invention will be described referring to FIG. 5. In the second embodiment, described is an example of generating images using a successive approximation reconstruction process including a forward projection process in which overlap between the adjacent pixels is taken into account. It is noted that repeated descriptions are omitted in the following description because the details of the back projection process in which overlap between the adjacent pixels is taken into account are the same as the first embodiment.

First, scanning conditions and reconstruction conditions are input from the input device 121 of the X-ray CT apparatus 1 before scanning an object. The scanning conditions and the reconstruction conditions are similar to the above first embodiment.

The image processing device 122 acquires projection data acquired by scanning, executes an image reconstruction method based on the above reconstruction conditions, and generates reconstruction images. In order to generate reconstruction images, the image processing device 122 first executes a filter correction three-dimensional back projection method including back projection taking into account overlap between adjacent pixels related to the present invention (the method used in the first embodiment).

Next, the image processing device 122 receives instruction input of whether or not to execute a successive approximation process.

After checking the reconstruction images generated by the above filter correction three-dimensional back projection method or the like, in a case where an operator determines that the reconstruction images have much noise or many artifacts resulting in diagnostic problems, the operator chooses to execute the successive approximation process via the input device 121. The image processing device 122 receives parameter settings for the successive approximation process from the operator.

The parameters of the successive approximation process include the maximum number of repetitions, convergence conditions (termination conditions), a prior probability weight (a coefficient determining a degree of smoothing), and the like. After the parameters of the successive approximation process are input and an execution instruction of the successive approximation process is input from the input device 121, the image processing device 122 starts the successive approximation process.

In the successive approximation process to be executed, the image processing device 122 first generates an initial image. The initial image may be an image reconstructed using the filter correction three-dimensional back projection method including back projection taking into account overlap between adjacent pixels as described in the first embodiment, and the other reconstruction methods may be used. It is noted that a constant-value image can be used for the initial value instead of a reconstruction image.

However, the number of repetitions until convergence in the successive approximation process varies according to a reconstruction method and a reconstruction filter to be used for initial image generation. In a case where there is much contradiction between projection data and forward projection data acquired by performing forward projection on the initial image, i.e. In a case where there is much contradiction between the projection data and the forward projection data because the initial image includes many artifacts, much distortion, or much noise, the number of repetitions until convergence is increased. Therefore, it is desirable to use the reconstruction method and the reconstruction filter so that forward projection data with less contradiction to projection data can be acquired.

Similarly, it is desirable to reduce noise and artifacts using an image quality improving filter to be used on projection data and image data during initial image generation.

The image processing device 122 performs a successive approximation process (successive approximation reconstruction) using forward projection and back projection that take into account overlap between adjacent pixels based on the acquired initial image. Thus, a successive approximation reconstructed image can be acquired. It is noted that parts other than the forward projection and the back projection in the successive approximation reconstruction are similar to the conventional successive approximation reconstruction method. Publicly known successive approximation reconstruction methods such as an ML (Most Likelihood) method, a MAP (Maximum a Posterior) method, a WLS (Weighted Least Squares) method, a PWLS (Penalized Weighted Least Squares) method, and a SIRT (Simultaneous Reconstruction Technique) method can be used as the successive approximation method.

Also, accelerating methods such as OS (Ordered Subset), SPS (Separable Paraboloidal Surrogate), and RAMLA (Row-Action Maximum Likelihood Algorithm) may be applied to these successive approximation methods.

Also, pixel overlap is taken into account in a back projection process and a forward projection process in successive approximation reconstruction. Because the back projection process that takes into account the pixel overlap was described in the first embodiment, the description is omitted, and the forward projection process will be described hereinafter.

In the forward projection of the present invention, the image processing device 122 sets a pixel size wider than a pixel interval similarly to back projection. This generates overlap between adjacent pixels. The image processing device 122 calculates size-dependent weights (pixel windows 2a to 2g: refer to FIGS. 2 and 3) according to an overlap amount between adjacent pixels and calculates an interpolation value to be assigned to beams using the size-dependent weights (pixel windows 2a to 2g).

Hereinafter, referring to the flow chart of FIG. 5, a procedure for calculating a pixel interpolation value by will be described by taking into account the overlap amount between adjacent pixels.

The processes in Steps S201 to S203 in the flow chart of FIG. 5 are similar to a case of the back projection (Steps S101 to S103 of FIG. 4) taking into account pixel overlap in the first embodiment.

That is, after a pixel size psx [mm] is determined by reconstruction conditions or the like set by an operator through the input device 121 or the like (Step S201), the image processing device 122 evaluates a pixel interval ppx using an effective visual field size FOV and a matrix size of a reconstruction image MATRIX or calculates a pixel window width pww and a pixel-size-dependent weight value pwtₖ from the pixel size psx and the pixel interval ppx using the above equations (1) to (5) (Steps S202 and S203).

Next, the image processing device 122 calculates an interpolation kernel g (Step S204) and calculates a pixel interpolation value by (Step S205). Hereinafter, calculation of the interpolation kernel g and the pixel interpolation value by will be described.

In a case of using a pixel size as a pixel interval and a beam size as a beam interval, a pixel interpolation value bvᵢ to be assigned to a beam bcᵢ is calculated by the following equations (9) and (10) by setting as follows: positions of pixel boundaries psⱼ and peⱼ of a pixel pcⱼ (j is a pixel index) on the common axis 4: P(psⱼ) and P(peⱼ), positions of beam boundaries bsᵢ and beᵢ of a beam bcᵢ (i is a beam index) on the common axis 4: P(bsᵢ) and P(beᵢ), pixels in which the beam boundaries P(bsⱼ) and P(beⱼ) on the common axis 4 are located: pcⱼₛ and pcⱼₑ, an interpolation kernel, i.e. a rate at which the pixel pcⱼ on the common axis 4 occupies the beam bc; (rate of the length on the common axis 4): g_{i, j}, and a pixel value located in a position j on the common axis 4: imgⱼ.

$$\begin{matrix}{xx}_{j} = {\sum }_{k = 0}^{pww - 1} {pwt}_{k} \cdot {img}_{j + psc + k} & ( 9 ) \\ {bv}_{i} {\sum }_{j = is}^{ie} {g}_{i , j} \cdot {xx}_{j} & ( 10 )\end{matrix}$$

The image processing device 122 assigns the above pixel interpolation value bvᵢ to the beam bcᵢ (Step S206).

As described above, a pixel size is set wider than a pixel interval in the second embodiment in order to perform a forward projection process and a back projection process by taking into account pixel overlap in a case where the image processing device 122 reconstructs images using a successive approximation method. It is desirable to perform a back projection process by taking into account the pixel overlap also in initial image generation in a successive approximation method.

Hence, noise increase can be suppressed in a case where a pixel size is large. Data usage inefficiency does not occur also in a case where a pixel size is larger than a pixel interval. Therefore, satisfactory image quality can be acquired even in a successive approximation reconstruction process that repeatedly performs a back projection and a forward projection. Consequently, occurrence of high-frequency errors such as moiré can be suppressed.

### Third Embodiment

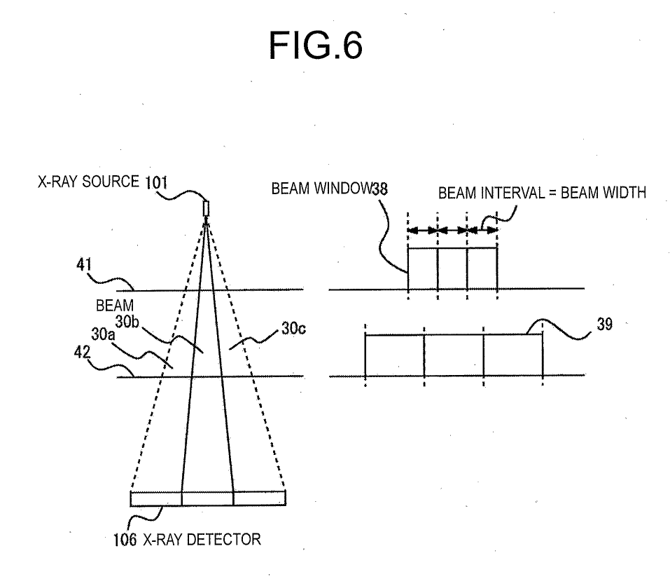

Next, a third embodiment of the present invention will be described referring to FIGS. 6 to 10.

Generally, in back projection by a distance-driven method, beam windows 38 and 39 of sizes according to distances from the X-ray source 101 to pixel positions 41 and 42 are set by matching beam intervals and beam widths of the respective beams 30a, 30b, and 30c to be radiated from the X-ray source 101 as illustrated in FIG. 6.

The adjacent beams 30a, 30b, and 30c are arranged continuously without being overlapped.

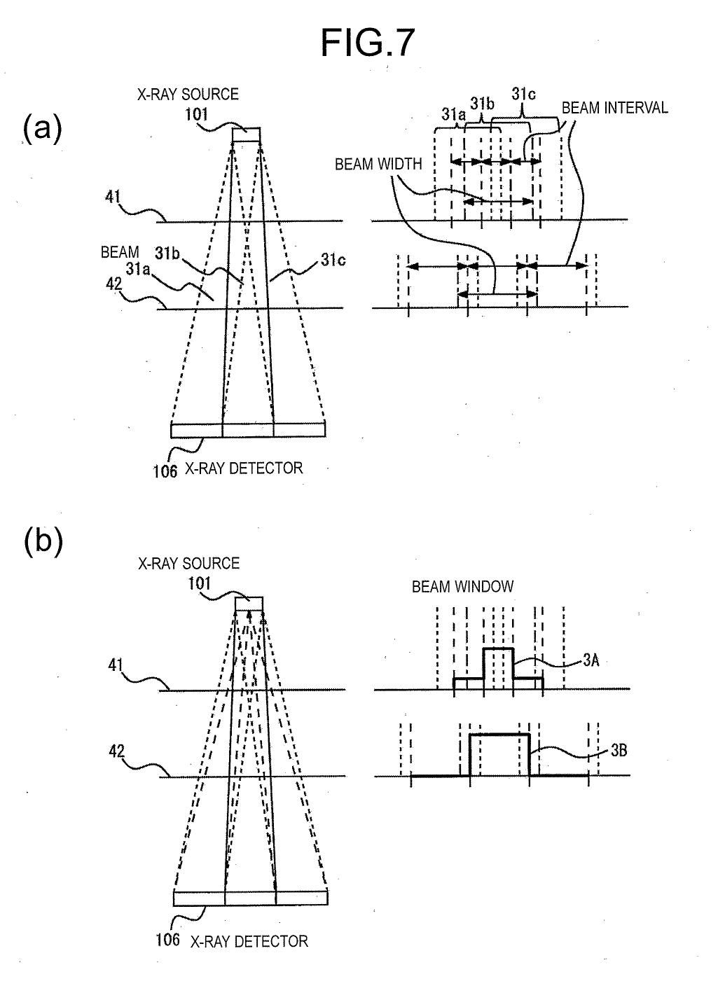

On the contrary to this, a beam to be irradiated from the X-ray source 101 realistically has a width. As illustrated in FIG. 7, the focus of the X-ray source 101 is not a point actually but has a certain size (area). Therefore, as illustrated in FIG. 7(a), beams 31a, 31b, and 31c having areas are emitted from the ray source, and overlap between the adjacent beams 31a, 31b, and 31c occurs in the pixel positions 41 and 42. In order to perform back projection or forward projection using the beams 30a, 30b, and 30c whose ray source is a point as illustrated above in FIG. 6 by taking into account the ray source size, a plurality of points of the ray source are set, calculation for the back projection or the forward projection is performed in all the points (the ray source), and then it is required to acquire an average value of the calculation results. Consequently, a calculation amount increases.

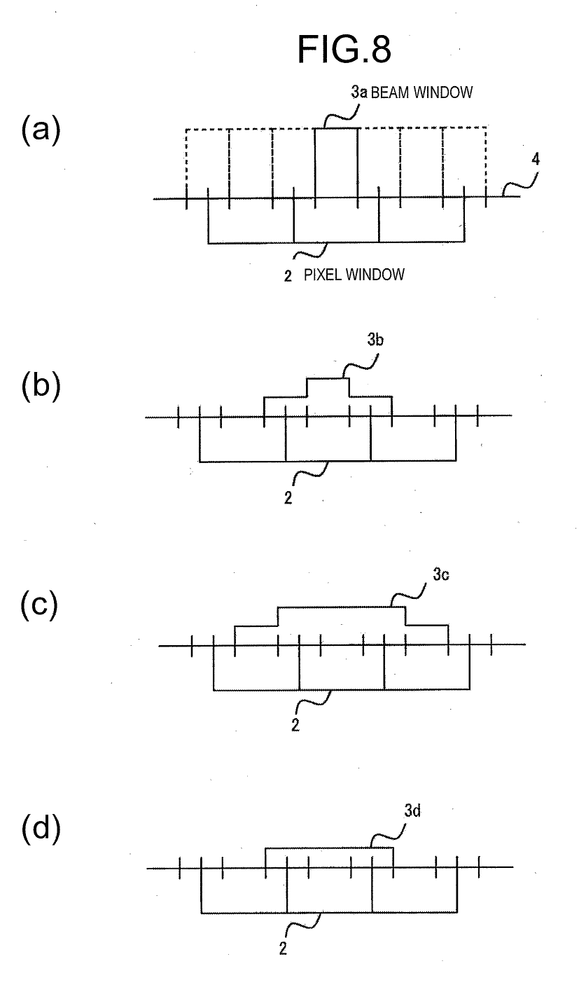

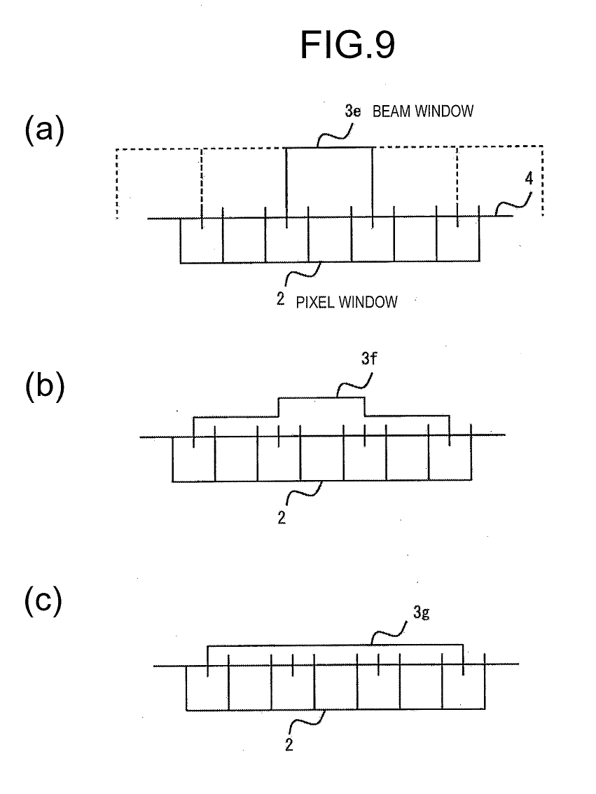

Therefore, in the third embodiment of the present invention, beams are irradiated from the X-ray source 101 with an area as illustrated in FIG. 7. The image processing device 122 sets a beam width wider than a beam interval and performs back projection by taking into account overlap between adjacent beams. Specifically, performed is a back projection process that sets a beam width wider than a beam interval and calculates a beam interpolation value to be assigned to each pixel using beam windows 3a to 3g (refer to FIGS. 8 and 9) with weight values according to the respective overlap amount of adjacent beams 31a, 31b, and 31c.

Also, because the overlap amount of the adjacent beams 31a, 31b, and 31c are different according to distances to pixel positions 41 and 42 from the X-ray source 101, the beam windows 3 to be applied are changed according to the overlap amount of the beams. For example, a beam window 3A whose width is double the beam interval is used as illustrated in the upper part of FIG. 7(b) in the pixel position 41 close to the X-ray source 101, and a beam window 3B whose width is equal to the beam interval is used because the overlap amount of the beams is reduced as illustrated in the lower part of FIG. 7(b) in the pixel position 42 distant from the X-ray source 101. It is noted that the beam windows 3a to 3g, 3A, and 3B are collectively referred to as the beam window 3 in the following description.

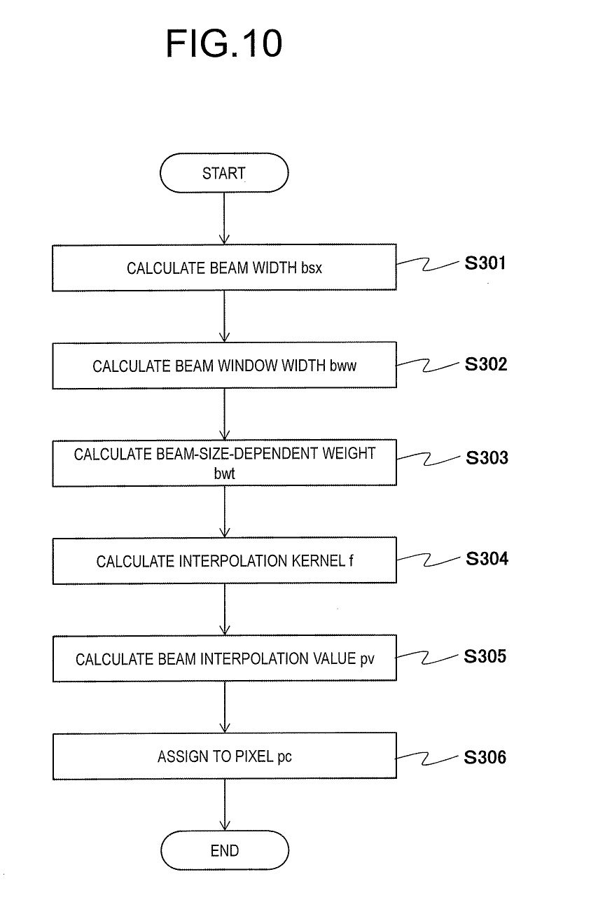

In the back projection in the third embodiment of the present invention, a beam window width bww is determined from a beam size (a beam width bsx) and a beam interval bpx. Additionally, a beam-size-dependent weight value bwtₖ is determined so that the sum of weight values (beam-size-dependent weight value bwtₖ) for which the adjacent beam windows 3 are overlapped and arranged is equal in each pixel position and a half-value width of the beam windows 3 is equal to the beam width. For example, by setting the beam windows 3a to 3g and the pixel window 2 as illustrated FIGS. 8 and 9, a value pv to be assigned to pixels is calculated using the procedure illustrated in the flow chart of FIG. 10.

The beam window 3 is a weight (size-dependent weight) to be used for calculating interpolation values to be assigned to pixels in a back projection process or to be assigned to projection (beams) in a forward projection process. The beam window 3 to be used is determined according to an overlap amount of adjacent beams. For example, a beam window to be used is changed according to a distance between the ray source and a pixel position. Also, a shape of the beam window 3 is defined by a width of the beam window 3 (beam window width bww) and a magnitude of the weight in each position (pixel region) in the width direction (beam-size-dependent weight value bwtₖ). The length in the vertical direction of each of the beam windows 3a to 3g illustrated in FIGS. 8 and 9 indicates a beam-size-dependent weight value bwtₖ. k is an index (a number that indicates the order of beam regions from the left, such as 0, 1, 2, . . . ) in the beam window 3. The beam regions are the respective regions in which beams were segmented at a beam interval.

FIG. 8 shows an example of arrangement of the beam window 3 and the pixel window 2 in a case where a beam size (beam width bsx) is set wider than a beam interval bpx and a beam window width bww is less than a pixel window width pww. (a) illustrates a shape of the beam window 3a in a case where the beam window width bww is equal to the beam interval bpx, (b) illustrates a shape of the beam window 3b in a case where the beam window width bww is set to double the beam interval bpx, (c) illustrates a shape of the beam window 3c in a case where the beam window width bww is set to quadruple the beam interval bpx, and (d) illustrates a shape of the beam window 3d in a case where the beam window width bww is set to triple the beam interval bpx.

Also, FIG. 9 illustrates arrangement of the pixel window 2 and the beam window 3 in a case where a beam size (beam width bsx) is set wider than a beam interval bpx and a beam window width bww is greater than a pixel window width pww. (a) illustrates a shape of the beam window 3e in a case where the beam window width bww is equal to the beam interval bpx, (b) illustrates a shape of the beam window 3f in a case where the beam window width bww is set to double the beam interval bpx, and (c) illustrates a shape of the beam window 3g in a case where the beam window width bww is set to triple the beam interval bpx.

It is noted that the beam windows 3a, 3b, . . . are collectively referred to as the beam window 3 in the following description.

Hereinafter, referring to the flow chart of FIG. 10, a procedure for calculating a beam interpolation value pv using the beam windows 3 illustrated in FIGS. 8 and 9 will be described.

As illustrated in the flow chart of FIG. 10, the image processing device 122 first calculates a beam size (beam width) bsx [mm] and a beam interval bpx [mm] (Step S301).

When a ray source size is fsx [mm]; a detector element size is dsx [mm]; a ray source-detector distance is SID [mm]; and a ray source-pixel distance is SPD [mm], a beam size (beam width) bsx [mm] in a pixel position is expressed by the following equation (11), and a beam interval bpx [mm] is expressed by the following equation (12).

$$\begin{matrix}bsx = fsx + \frac{( dsx - fsx ) \cdot SPD}{SID} & ( 11 ) \\ bpx = \frac{dsx \cdot SPD}{SID} & ( 12 )\end{matrix}$$

After the beam size bsx [mm] in a pixel position and the beam interval bpx [mm] are calculated (Step S201), the image processing device 122 evaluates a beam window width bww [channel] in the pixel position using the following equation (13) (Step S202).

$$bww = ⌈ \frac{bsx - bpx}{2 \cdot bpx} ⌉ \cdot 2 + 1 \qquad ( 13 )$$

It is noted that a beam window center position bwc of a beam window width bww is expressed by the equation (14), and a beam window end position bwe of the beam window center position bwc is expressed by the equation (15).

$$\begin{matrix}bwc = \frac{bww - 1}{2} & ( 14 ) \\ bwe = \pm ⌈ \frac{bsx}{2 \cdot bpx} ⌉ & ( 15 )\end{matrix}$$

Next, the image processing device 122 segments beams at a beam interval in order to determine a beam-size-dependent weight value bwtₖ in each of the segmented beam regions. That is, the following equation (16) is used for calculating a beam-size-dependent weight value bwtₖ of the k-th region from the left end pixel (pixel of k=0) in the beam window 3 (Step S303).

$${bwt}_{k} = \{ \begin{matrix}\frac{\frac{bsx - bpx}{2 \cdot bpx} - ⌊ \frac{bsx - bpx}{2 \cdot bpx} ⌋}{bsx} & if k = bwc + bwe \\ \frac{1}{bsx} & if bwc - bwe < k < bwc + bwe\end{matrix} \qquad ( 16 )$$

A leading pixel position bsc of the beam window 3 is expressed by the following equation (17).

$$bsc = - ⌈ \frac{bsx - bpx}{2 \cdot bpx} ⌉ \qquad ( 17 )$$

Next, the image processing device 122 calculates an interpolation kernel f (Step S304) and calculates a beam interpolation value pv (Step S305). Hereinafter, calculation of the interpolation kernel f and the beam interpolation value pv will be described.

In a case of using a pixel size as a pixel interval and a beam size as a beam interval, a beam interpolation value pvⱼ to be assigned to a pixel pcⱼ is calculated by the following equations (18) and (19) by setting as follows:

positions of pixel boundaries psⱼ and peⱼ of a pixel pcⱼ (j is a pixel index) on the common axis 4: P(psⱼ) and P(peⱼ);

positions of beam boundaries bsᵢ and beᵢ of a beam bcᵢ (i is a beam index) on the common axis 4: P(bsᵢ) and P(beᵢ);

beams in which the beam boundaries P(bsᵢ) and P(beᵢ) on the common axis 4 are located: bcⱼₛ and bcⱼₑ;

an interpolation kernel, i.e. a rate at which the beam bcᵢ on the common axis 4 occupies the pixel pcⱼ (rate of the length on the common axis 4): f_{i, j}; and

a projection value located in a position i on the common axis 4: rawᵢ.

$$\begin{matrix}{yy}_{i} = {\sum }_{k = 0}^{bww - 1} {bwt}_{k} \cdot {raw}_{i + bsc + k} & ( 18 ) \\ {pv}_{j} = {\sum }_{i = js}^{je} {f}_{i , j} \cdot {yy}_{i} & ( 19 )\end{matrix}$$

The image processing device 122 assigns the above beam interpolation value pvⱼ to a pixel pcⱼ (Step S306).

As described above, in the third embodiment, a back projection process is performed by taking into account overlap of adjacent beams in the back projection process or the like in a filter correction three-dimensional back projection method or a successive approximation method.

Hence, satisfactory image quality with excellent data usage efficiency can be acquired without image quality deterioration caused by data usage inefficiency.

Also, a relationship between a beam size and a beam interval (beam overlapping degree) is changed according to a distance from the ray source to a target pixel, a ray source size, a detection element size, and a distance between the ray source and the detection element. Therefore, the sequential calculation can acquire results at a high speed by taking into account the ray source size and the detection element size.

By applying the back projection process of the third embodiment to a beam whose ray source is not a point but has a size (area), the back projection process can be performed at a high speed while improving the model accuracy during successive approximation reconstruction.

### Fourth Embodiment

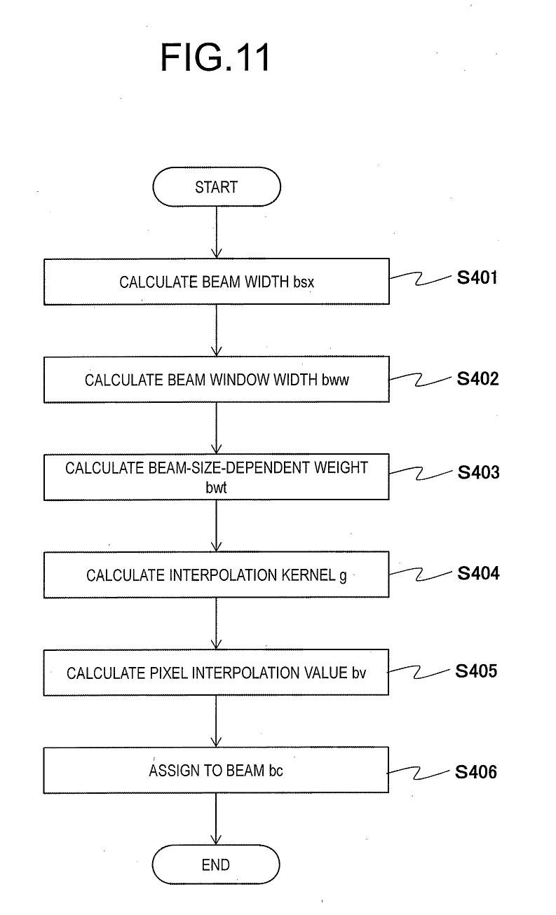

Next, a fourth embodiment of the present invention will be described referring to FIG. 11. In the fourth embodiment, a forward projection method will be described by taking into account overlap between adjacent beams. The overlap between adjacent beams is similar to the third embodiment (refer to FIG. 7), and the description is omitted.

In the forward projection taking into account the overlap between adjacent beams, a beam window width bww is determined from a beam size (beam width bsx) and a beam interval bpx similarly to the third embodiment (a case of the back projection). Additionally, a beam-size-dependent weight value bwtₖ is determined so that the sum of beam-size-dependent weight values bwtₖ when the adjacent beam windows 3 are overlapped and arranged is equal in each pixel position and so that a half-value width of the beam window 3 is equal to a beam width.

For example, the beam windows 3 and the pixel windows 2 illustrated in FIGS. 8 and 9 are set in order to calculate a value by to be assigned to a beam using the procedure illustrated in FIG. 11.

Hereinafter, referring to the flow chart of FIG. 11, a procedure for calculating a pixel interpolation value by using the beam windows 3 illustrated in FIGS. 8 and 9 will be described.

Processes of Steps S401 to S403 in the flow chart of FIG. 11 are similar to a case of the back projection of the third embodiment (Steps S301 to S303 of FIG. 10).

That is, the image processing device 122 calculates a beam size (beam width) bsx and a beam interval bpx in a pixel position from a ray source size fsx, a detector element size dsx, a ray source-detector distance SID, and a ray source-pixel distance SPD using the above equations (11) and (12). Also, a beam window width bww is calculated based on the beam interval bpx and the beam size bsx (the equation (13)). Additionally, the image processing device 122 calculates a beam-size-dependent weight value bwtₖ similarly to the above equation (16).

Next, the image processing device 122 calculates an interpolation kernel g (Step S404) and calculates a pixel interpolation value by (Step S405). Hereinafter, calculation of the interpolation kernel g and the pixel interpolation value by will be described.

In a case of using a pixel size as a pixel interval and a beam size as a beam interval, a pixel interpolation value bvᵢ to be assigned to a beam bcᵢ is calculated by the following equations (20) and (21) by setting as follows:

positions of pixel boundaries psⱼ and peⱼ of a pixel pcⱼ (j is a pixel index) on the common axis 4: P(psⱼ) and P(peⱼ);

positions of beam boundaries bsᵢ and beᵢ of a beam bcᵢ (i is a beam index) on the common axis 4: P(bsᵢ) and P(beᵢ);

pixels in which the beam boundaries P(bsᵢ) and P(beᵢ) on the common axis 4 are located: pcᵢₛ and pcᵢₑ;

an interpolation kernel, i.e. a rate at which the pixel pcⱼ on the common axis 4 occupies the beam bcᵢ (rate of the length on the common axis 4): g_{i, j}; and

a pixel value located in a position j on the common axis 4: imgⱼ.

$$\begin{matrix}{xx}_{i} = {\sum }_{j = is}^{ie} {g}_{i , j} \cdot {img}_{j} & ( 20 ) \\ {bv}_{i} = {\sum }_{k = 0}^{bww - 1} {bwt}_{k} \cdot {xx}_{i + bsc + k} & ( 21 )\end{matrix}$$

The image processing device 122 assigns the above pixel interpolation value bvᵢ to a beam bcᵢ (Step S406).

As described above, in the fourth embodiment, a forward projection method is performed by taking into account overlap of adjacent beams in the forward projection process or the like during image reconstruction by a successive approximation method. This can perform the forward projection by taking into account a ray source size and acquire satisfactory image quality with excellent data usage efficiency without image quality deterioration caused by data usage inefficiency. Also, a relationship between a beam size and a beam interval (beam overlapping degree) is changed according to a distance from the ray source to a target pixel, a ray source size, a detection element size, and a distance between the ray source and the detection element. Therefore, the sequential calculation can acquire results at a high speed by taking into account the ray source size and the detection element size.

By applying the forward projection process of the fourth embodiment to a beam whose ray source is not a point but has a size (area), the forward projection process can be performed at a high speed while improving the model accuracy during successive approximation reconstruction.

[Fifth Element]

Next, described will be a back projection method taking into account both overlap between adjacent beams and overlap between adjacent pixels as a fifth embodiment of the present invention.

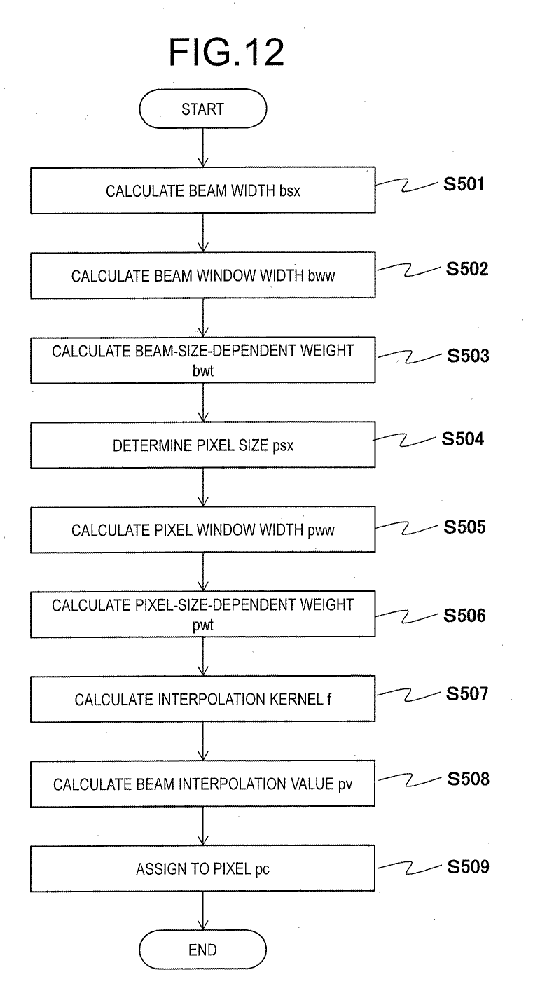

In the back projection method of the fifth embodiment, the pixel windows 2 and the beam windows 3 illustrated in FIGS. 2 and 3 and FIGS. 8 and 9 are used similarly to the first and third embodiments. Hereinafter, referring to the flow chart of FIG. 12, described will be a procedure for calculating a beam interpolation value pv in the back projection taking into account overlap between adjacent beams and overlap between adjacent pixels.

The image processing device 122 first calculates a beam size (beam width) bsx, a beam interval bpx, a beam window width bww, and a beam-size-dependent weight value bwtₖ in a pixel position similarly to a case of the back projection (Steps S301 to S303 of FIG. 10) taking into account beam overlap of the third embodiment (Steps S501 to S503). That is, the image processing device 122 calculates a beam size (beam width) bsx and a beam interval bpx in a pixel position from a ray source size fsx, a detector element size dsx, a ray source-detector distance SID, and a ray source-pixel distance SPD using the above equations (11) and (12). Also, the beam window width bww is calculated based on the beam interval bpx and the beam size bsx (the equation (13)). Additionally, the image processing device 122 calculates the beam-size-dependent weight value bwtₖ similarly to the above equations (14) to 16).

Also, the image processing device 122 calculates a pixel size psx, a pixel interval ppx, a pixel window width pww, a pixel-size-dependent weight value pwtₖ similarly to a case of back projection (Steps S101 to S103 of FIG. 4) taking into account pixel overlap in the first embodiment (Steps S504 to S506). The pixel size psx [mm] is determined under reconstruction conditions or the like set by an operator through the input device 121, and the pixel interval ppx and the pixel window width pww are calculated using an effective visual field size FOV, a matrix size of a reconstruction image MATRIX, and the like respectively from the equations (1) and (2). A beam-size-dependent weight value bwtₖ is calculated using the above equations (3) to (5).

Next, the image processing device 122 calculates an interpolation kernel f (Step S507) and calculates a beam interpolation value pv (Step S508). Hereinafter, calculation of the interpolation kernel f and the beam interpolation value pv will be described.

In a case of using a pixel size as a pixel interval and a beam size as a beam interval, a value pvⱼ to be assigned to a pixel pcⱼ is calculated by the following equations (22), (23) and (24) by setting as follows: positions of pixel boundaries psⱼ and peⱼ of a pixel pcⱼ (j is a pixel index) on the common axis 4: P(psⱼ) and P(peⱼ), positions of beam boundaries bsᵢ and beᵢ of a beam bcᵢ (i is a beam index) on the common axis 4: P(bsᵢ) and P(beᵢ), beams in which the pixel boundaries P(psᵢ) and P(peᵢ) on the common axis 4 are located: bcⱼₛ and bcⱼₑ, an interpolation kernel, i.e. a rate at which the beam bcᵢ on the common axis 4 occupies the pixel pcⱼ (rate of the length on the common axis 4): f_{i, j}, and a projection value located in a position i on the common axis 4: rawⱼ.

$$\begin{matrix}{yy}_{i} = {\sum }_{k = 0}^{bww - 1} {bwt}_{k} \cdot {raw}_{i + bsc + k} & ( 22 ) \\ {zz}_{j} = {\sum }_{i = js}^{je} {f}_{i , j} \cdot {yy}_{i} & ( 23 ) \\ {pv}_{j} = {\sum }_{k = 0}^{pww - 1} {pwt}_{k} \cdot {zz}_{j + psc + k} & ( 24 )\end{matrix}$$

The image processing device 122 assigns the above beam interpolation value pvⱼ to the pixel pcⱼ (Step S509).

As described above, in the fifth embodiment, both overlap between adjacent beams and overlap between adjacent pixels are taken into account in the back projection process. This can use data uniformly and acquire satisfactory image quality with excellent data usage efficiency without image quality deterioration caused by data usage inefficiency. Consequently, occurrence of high-frequency errors such as moiré can be suppressed.

The back projection process of the fifth embodiment can be applied during image reconstruction by a filter correction three-dimensional back projection method, during image reconstruction for determining whether or not to reconstruct images by a successive approximation method as described in the second embodiment, during image generation by the successive approximation method, or the like.

### Sixth Embodiment

Next, as a sixth embodiment of the present invention, a forward projection method taking both overlap between adjacent beams and overlap between adjacent pixels will be described.

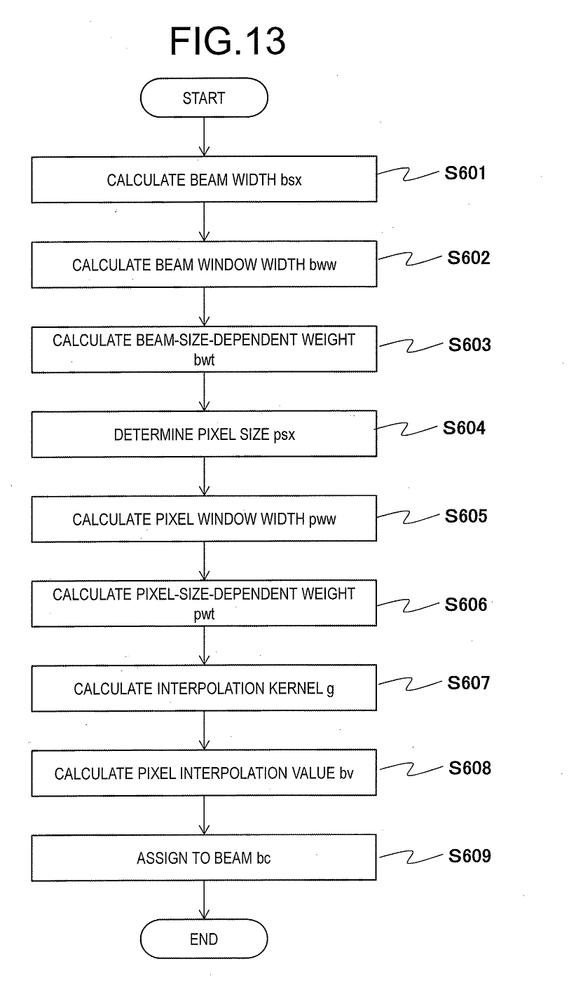

In the forward projection method of the sixth embodiment, the pixel windows 2 and the beam windows 3 illustrated in FIGS. 2 and 3 and FIGS. 8 and 9 are used similarly to the second and fourth embodiments. Hereinafter, referring to the flow chart of FIG. 13, described will be a procedure for calculating a pixel interpolation value by in the forward projection taking into account overlap between adjacent beams and overlap between adjacent pixels.

First, the image processing device 122 calculates a beam size (beam width) bsx, a beam interval bpx, a beam window width bww, and a beam-size-dependent weight value bwtₖ in a pixel position similarly to a case of the forward projection (Steps S401 to S403 of FIG. 11) taking into account beam overlap of the fourth embodiment (Steps S601 to S603). That is, the image processing device 122 calculates a beam size (beam width) bsx and a beam interval bpx in a pixel position from a ray source size fsx, a detector element size dsx, a ray source-detector distance SID, and a ray source-pixel distance SPD using the above equations (11) and (12). Also, the beam window width bww is calculated based on the beam interval bpx and the beam size bsx (the equation (13)). Additionally, the image processing device 122 calculates the beam-size-dependent weight value bwtₖ similarly to the above equations (14) to (16).

Also, the image processing device 122 calculates a pixel size psx, a pixel interval ppx, a pixel window width pww, a pixel-size-dependent weight value pwtₖ similarly to a case of forward projection (Steps S201 to S203 of FIG. 5) taking into account pixel overlap in the second embodiment (Steps S604 to S606). The pixel size psx [mm] is determined under reconstruction conditions or the like set by an operator through the input device 121, and the pixel interval ppx and the pixel window width pww are calculated using an effective visual field size FOV, a matrix size of a reconstruction image MATRIX, and the like respectively from the equations (1) and (2). A pixel-size-dependent weight value pwtₖ is calculated using the above equations (3) to (5).

Next, the image processing device 122 calculates an interpolation kernel g (Step S607) and calculates a pixel interpolation value by (Step S608). Hereinafter, calculation of the interpolation kernel g and the beam interpolation value by will be described.

In a case of using a pixel size as a pixel interval and a beam size as a beam interval, a value bvᵢ to be assigned to a pixel bcᵢ is calculated by the following equations (25), (26) and (27) by setting as follows: positions of pixel boundaries psⱼ and peⱼ of a pixel pcⱼ (j is a pixel index) on the common axis 4: P(psⱼ) and P(peⱼ), positions of beam boundaries bsᵢ and beᵢ of a beam bc; (i is a beam index) on the common axis 4: P(bsᵢ) and P(beᵢ), pixels in which the beam boundaries P(bsᵢ) and P(beᵢ) on the common axis 4 are located: pcᵢₛ and pcᵢₑ, an interpolation kernel, i.e. a rate at which the pixel pcⱼ on the common axis 4 occupies the beam bcᵢ (rate of the length on the common axis 4): g_{i, j}, and a pixel value located in a position j on the common axis 4: imgⱼ.

$$\begin{matrix}{xx}_{j} = {\sum }_{k = 0}^{pww - 1} {pwt}_{k} \cdot {img}_{j + psc + k} & ( 25 ) \\ {zz}_{i} = {\sum }_{j = is}^{ie} {g}_{i , j} \cdot {xx}_{j} & ( 26 ) \\ {bv}_{i} = {\sum }_{k = 0}^{bww - 1} {bwt}_{k} \cdot {zz}_{i + bsc + k} & ( 27 )\end{matrix}$$

The image processing device 122 assigns the above pixel interpolation value bvᵢ to the beam bcᵢ (Step S609).

As described above, in the sixth embodiment, a forward projection process is performed by taking into account both overlap between adjacent beams and pixel overlap in a forward projection method. This can use data uniformly and acquire satisfactory image quality with excellent data usage efficiency without image quality deterioration caused by data usage inefficiency. Consequently, occurrence of high-frequency errors such as moiré can be suppressed.

The back projection process of the sixth embodiment can be applied during image generation by a successive approximation method.

### Seventh Embodiment

In the seventh embodiment, described will be methods for back projection and forward projection taking into account a beam dose distribution (electronic density distribution) and sensitivity of the X-ray detector 106.

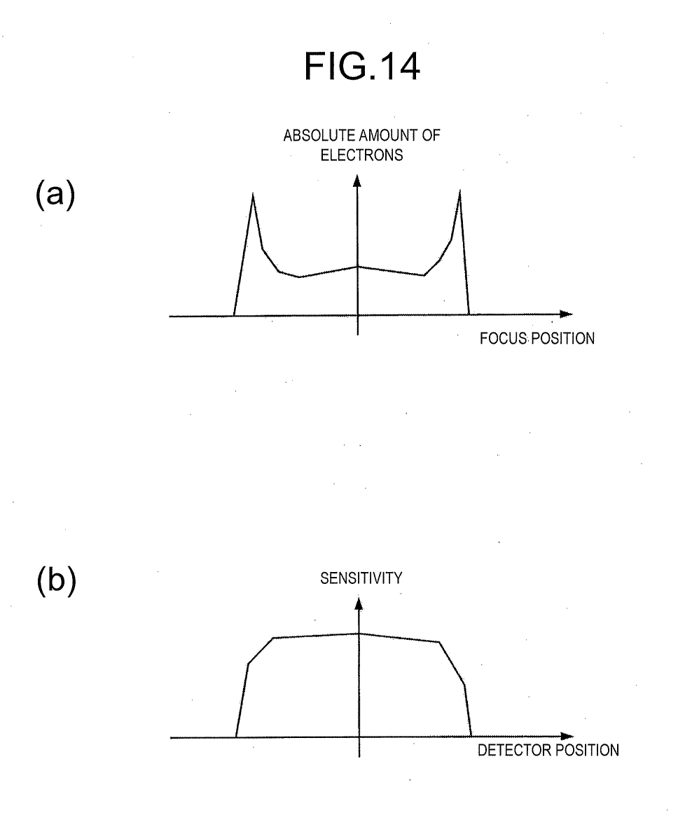

FIG. 14(a) illustrates a dose distribution (electronic density distribution) at the X-ray source 101, and FIG. 14(b) illustrates a sensitivity distribution of the X-ray detector 106.

As illustrated in FIG. 7, the focus of the X-ray source 101 is not a point actually but has a certain size (area). A dose of beams (electronic density) to be irradiated from the plane is characterized by differences according to the focal point as illustrated in FIG. 14(a). As illustrated in FIG. 14(b), the sensitivity of the X-ray detector 106 also differs according to the detector position.

Therefore, in the seventh embodiment, the image processing device 122 superimposes a dose distribution function or a detector sensitivity distribution function illustrated in FIG. 14 on the beam windows 3 (FIGS. 8 and 9) exemplified in the third and fourth embodiments. Then, the image processing device 122 standardizes the beam windows 3 after superimposing the dose distribution function or the detector sensitivity distribution function so that the sum of weight values for which weighting was performed between adjacent beams is equal in each pixel position in order to acquire modified beam windows. The image processing device 122 performs forward projection or back projection of any of the third to sixth embodiments using the above modified beam windows during image reconstruction.

Hence, image reconstruction can be performed in a state where intensity of X-ray beams to be irradiated from the X-ray source with areas is modified so as to be equal.

Although suitable embodiments of the present invention are described above, the present invention is not limited to the above embodiments. For example, although one-dimensional processes are exemplified in each of the above embodiments, the present invention may be applied to a case of calculating an interpolation value for projection data acquired a two-dimensional detector. In this case, the interpolation value is first calculated in the channel direction, and then a final interpolation value can be acquired by calculating the interpolation value in the column direction.

Also, it is desirable that the interpolation value is not reduced by extrapolating data in the data end, adjusting a weight value of a size-dependent weight, or the like. The present invention can be applied also to back/forward projection of a fan beam method and back/forward projection of a parallel beam method. Additionally, the data processing methods of the present invention can be applied to image reconstruction in various X-ray CT apparatus or the like using a single-slice detector, a multi-slice detector, or a flat-panel detector.

Although both forward projection and back projection taking into account a pixel size and a ray source size are performed for successive approximation reconstruction in each of the above embodiments, only either of the forward projection or the back projection taking into account a pixel size and a ray source size may be used.

Additionally, it is apparent that a person skilled in the art could arrive at various modified examples or amended examples within the scope of the technical ideas disclosed in the present invention, and it is understood that these naturally belong to the technical scope of the present invention.

### REFERENCE SIGNS LIST

1: X-ray CT apparatus, 100: scan gantry unit, 101: X-ray source, 102: rotary disk, 106: X-ray detector, 120: operation console, 121: input device, 122: image processing device (data processing device), 123: storage device, 124: system controller, 125: display device, 2 and 2a to 2g: pixel window (pixel-size-dependent weight), 3; 3a to 3g; 3A; and 3B: beam window (beam-size-dependent weight), 4: common axis, 41 and 42: pixel position, 5: pixel

1. A data processing method,
wherein a beam size is set wider than a beam interval or a pixel size is set wider than a pixel interval in order to calculate an interpolation value to be assigned to a beam or a pixel using a size-dependent weight according to an overlap amount of adjacent beams or an overlap amount of adjacent pixels in a forward projection process or a back projection process to be executed by a data processing device.

2. The data processing method according to claim 1,
wherein the data processing device performs the forward projection process or the back projection process including steps of:
calculating a width of the size-dependent weight based on the pixel size and the pixel interval;
calculating a weight value of the size-dependent weight in each of pixel regions in which pixels were segmented at the pixel interval;
calculating an interpolation value to be assigned to beams or pixels based on the size-dependent weight and an interpolation kernel; and
assigning the calculated interpolation value to the beams or the pixels.

3. The data processing method according to claim 2,
wherein the pixel size is a slice thickness of an reconstruction image, and the pixel interval is a slice interval of the reconstruction image.

4. The data processing method according to claim 1,
wherein the data processing device performs the forward projection process or the back projection process including steps of:
calculating a width of the size-dependent weight based on the beam size and the beam interval;
calculating a weight value of the size-dependent weight in each of beam regions in which beams were segmented at the beam interval;
calculating an interpolation value to be assigned to beams or pixels based on the size-dependent weight and an interpolation kernel; and
assigning the calculated interpolation value to the beams or the pixels.

5. The data processing method according to claim 4,
wherein a step of changing a relationship between the beam size and the beam interval according to a distance from the ray source to a target pixel, a ray source size, a detector element size, and a distance between the ray source and the detector is further included.

6. The data processing method according to claim 1,
wherein a weight value of the size-dependent weight is calculated so that the sum of the size-dependent weights is equal in each pixel.

7. The data processing method according to claim 4,
wherein a step of obtaining a modified size-dependent weight by superimposing a function indicating a dose distribution in the ray source or a detector sensitivity distribution on the size-dependent weights and standardizing so that the sum of the superimposed size-dependent weights is equal between adjacent beams is further included, and
an interpolation value to be assigned to beams or pixels is calculated using the modified size-dependent weight.

8. A data processing device comprising:
a setting unit that sets a beam size wider than a beam interval or a pixel size wider than a pixel interval in a forward projection process or a back projection process; and
a calculation unit that calculates an interpolation value to be assigned to beams or pixels using a size-dependent weight according to an overlap amount of adjacent beams or an overlap amount of adjacent pixels.

9. An X-ray CT apparatus including the data processing device according to claim 8.

10. The X-ray CT apparatus comprising:
an X-ray source that irradiates X-rays from the focus with an area;
an X-ray detector that is disposed opposite to the X-ray source and detects X-rays transmitted through an object;
a data acquisition device that acquires transmission X-rays detected by the X-ray detector; and
an image processing device that obtains the transmission X-rays and executes an image reconstruction process that includes a process for setting a beam size wider than a beam interval in a forward projection process or a back projection process to reconstruct an image based on the obtained transmission X-rays and calculating an interpolation value to be assigned to beams or pixels using a size-dependent weight according to an overlap amount of adjacent beams.
